(19) United States
(12) Patent Application Publication
(10) Pub. No.: US 2026/0254284 A1
(43) Pub. Date: Aug. 27, 2026

(54) PHOTO-ELECTRIC COOPERATIVE INTERVENTION ORTHOKERATOLOGY LENS FOR MYOPIA IMPROVEMENT

(71) Applicant: University of Electronic Science and Technology of China, Chengdu (CN)

(72) Inventors: Yuan LIN, Chengdu (CN); Guang YAO, Chengdu (CN); Xingyi GAN, Chengdu (CN); Yi SHI, Chengdu (CN); Lingxi JIANG, Chengdu (CN); Min GAO, Chengdu (CN); Taisong PAN, Chengdu (CN)

(21) Appl. No.: 19/538,917

(22) Filed: Feb. 12, 2026

(30) Foreign Application Priority Data
Feb. 21, 2025 (CN) 202510196167.8

(51) Int. Cl. H02J 50/80; A61F 2/16; H02J 50/10; H02J 50/12

(52) U.S. Cl. CPC H02J 50/80; A61F 2/1624; H02J 50/10; H02J 50/12

(57) ABSTRACT

The invention provides a photo-electric cooperative intervention orthokeratology lens for myopia improvement, which belongs to the technical field of biomedical devices. The orthokeratology lens comprises an external transmitting terminal and an internal receiving terminal; the two terminals realize passive supply of the light field and electric field of the internal receiving terminal through electromagnetic coupling. The internal receiving terminal is composed of a light intervention unit and an electric field intervention unit. The light intervention unit is built-in with a red light LED to achieve red light intervention, and the electric field intervention unit generates a potential difference to form an electric field. By integrating the circuit with the orthokeratology lens, the invention accelerates myopia improvement and reduces the discomfort and risks caused by long-term wearing of orthokeratology lenses.

### CROSS-REFERENCE TO RELATED APPLICATIONS

Pursuant to 35 U.S.C.§ 119 and the Paris Convention Treaty, this application claims foreign priority to Chinese Patent Application No. 202510196167.8 filed Feb. 21, 2025, the contents of which, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, MA 02142.

### BACKGROUND

The disclosure relates to the field of biomedical devices, and more particularly to a photo-electric cooperative intervention orthokeratology lens for myopia improvement.

At present, myopia has become one of the major global public health challenges, and its prevalence rate among children and adolescents is rising rapidly. Studies have shown that the global prevalence rate of myopia among children and adolescents is about 30.5%, which has increased significantly over the past 30 years, rising from about one-quarter to one-third of the population: between 1990 and 2000, the global prevalence rate of myopia among children and adolescents was 24.3%; it reached 25.3% from 2001 to 2010; it further increased to 29.7% from 2011 to 2019; and during the period from 2020 to 2023, the myopia rate has surged dramatically, with the myopia rate among adolescents reaching as high as 54%.

Orthokeratology lenses are an effective non-surgical vision correction method, particularly suitable for myopia patients. Compared with laser surgery, orthokeratology lenses have lower risks of use and can slow down the progression of myopia in children and adolescents to a certain extent. However, once the wearing is discontinued, the cornea will gradually revert to its original state, which is reversible. Meanwhile, orthokeratology lenses also have some drawbacks; for example, they have to be worn for extended periods of time, which not only greatly increases the risk of infection for the wearer, but also causes a lot of discomfort.

### SUMMARY

To solve the aforesaid problems, the disclosure provides a photo-electric cooperative intervention orthokeratology lens for myopia improvement. The photo-electric cooperative intervention orthokeratology lens comprises an external transmitting terminal and an internal receiving terminal. The external transmitting terminal and an internal receiving terminal realize passive supply of the light field and electric field of the internal receiving terminal through electromagnetic coupling. The internal receiving terminal comprises a light intervention unit and an electric field intervention unit. The light intervention unit is built-in with a red light LED to achieve red light intervention, and the electric field intervention unit generates a potential difference to form an electric field.

The photo-electric cooperative intervention orthokeratology lens comprises an external power supply, an external transmitting terminal, an internal receiving terminal, and an orthokeratology lens body. The internal receiving terminal is disposed on an outer surface of the orthokeratology lens body, and an inner surface of the orthokeratology lens body is configured to contact an eyeball. The external transmitting terminal is integrated on an eyeglass frame. The external power supply is connected to the external transmitting terminal. The internal receiving terminal comprises a light intervention unit and an electric field intervention unit. The light intervention unit is configured to provide red light intervention, and the electric field intervention unit is configured to provide an electric field. The internal receiving terminal is electromagnetically coupled with the external transmitting terminal to provide wireless power supply to the light intervention unit and the electric field intervention unit.

In a class of this embodiment, electromagnetic coupling operates at a resonant frequency band of 40–50 MHz.

In a class of this embodiment, the light intervention unit comprises a first metal coil, a first chip capacitor, and a red surface-mount device light-emitting diode (hereinafter referred to as “red SMD LED”). The first metal coil comprises two concentric arcs and a first rectangular metal segment connecting the two concentric arcs. The first chip capacitor is connected in parallel with the red SMD LED, the first chip capacitor and the red SMD LED are disposed between unconnected ends of the two concentric arcs, and two ends of the first chip capacitor and the red SMD LED are respectively connected to the two concentric arcs to form a series resonant circuit.

In a class of this embodiment, red light emitted by the red SMD LED has a wavelength range of 650 nm–680 nm.

In a class of this embodiment, a power density of the red light LED is 0.1–2.0 mW/cm².

In a class of this embodiment, the electric field intervention unit comprises a first electrode, a second electrode, a second metal coil, a second chip capacitor, and a rectifier. The second metal coil has the same structure as the first metal coil and is symmetrically arranged with the first metal coil to form a circular shape; the second chip capacitor is connected in series between the unconnected ends of the two concentric arcs of the second metal coil; the first electrode comprises a first metal circle having a first opening, and a first end of the first opening is connected to an unconnected end of an inner arc of the second metal coil through a second rectangular metal segment; a circle formed by the first metal coil and the second metal coil is concentric with the first electrode; and the second electrode comprises a second metal circle having a second opening and an electrode wire connected to a first end of the second opening; a second end of the electrode wire is connected to a first end of the rectifier; and a second end of the rectifier is connected to an unconnected end of an outer arc of the second metal coil.

In a class of this embodiment, the second electrode comprises an indium–tin oxide (ITO) transparent electrode material.

In a class of this embodiment, the external transmitting terminal comprises a first patterned metal layer, a third chip capacitor, and a fourth chip capacitor. The first patterned metal layer comprises a third metal circle having a third opening, a semicircular metal strip, and a first metal arc segment, and a second metal arc segment; the first metal arc segment and the second metal arc segment are of equal length; the first metal arc segment and the second metal arc segment are disposed adjacent to each other; the semicircular metal strip is disposed on a first side of a diameter of the third metal circle, and the first metal arc segment and the second metal arc segment are disposed on a second side of the diameter; a center of the semicircular metal strip is metallically connected to the third metal circle; a first end of the first metal arc segment is connected to a first open end of the third metal circle through a third rectangular metal segment; and a first end of the second metal arc segment is connected to a second open end of the third metal circle through a fourth rectangular metal segment; two ends of the third chip capacitor are respectively connected to the first open end and second open end of the third metal circle; and a first end of the fourth chip capacitor is connected to the third metal circle and a second end of the fourth chip capacitor is connected to the external power supply.

In a class of this embodiment, the external power supply comprises a function signal generator.

In a class of this embodiment, the first chip capacitor and the second chip capacitor have a same capacitance value, and the capacitance values of the first chip capacitor, the third chip capacitor and the fourth chip capacitor are different from each other to enable electromagnetic coupling between the external transmitting terminal and the internal receiving terminal.

In a class of this embodiment, the capacitance value of the first chip capacitor is 120–680 pF; the capacitance value of the second chip capacitor is 120–680 pF; the capacitance value of the third chip capacitor is 1–10 nF; and the capacitance value of the fourth chip capacitor is 10–100 pF.

In a class of this embodiment, the first metal coil and the second metal coil are formed by laser cutting copper foil.

In a class of this embodiment, a thickness of the copper foil is 10–30 μm.

In summary, by adopting the above technical solution, the disclosure has the following beneficial effects.

By introducing electromagnetic coupling between the external transmitting terminal and the internal receiving terminal, designing the patterned structures of the internal receiving terminal and the external transmitting terminal, arranging the third chip capacitor and the fourth chip capacitor at the external transmitting terminal, disposing the first chip capacitor and the second chip capacitor at the internal receiving terminal, and supplemented by the rectifier, the disclosure enables the internal receiving terminal and the external transmitting terminal to have the same resonant frequency, which is controlled within the range of 40–50 MHz, thereby realizing LED illumination and electric field generation for intervention therapy and stimulation treatment. The LED adopts red light with a specific wavelength, which can penetrate to the fundus oculi and be absorbed by retinal tissues, potentially promoting the blood supply of the choroid and increasing the thickness of the choroid to alleviate myopia. The electric field assists the orthokeratology lens in guiding the stress transmission direction of corneal tissues under pressure, reducing the uneven deformation of local tissues, making the adjustment of corneal morphology more uniform and efficient, and reducing the risk of discomfort or complications caused by uneven shaping. By integrating the circuit with the orthokeratology lens, the disclosure accelerates myopia improvement and reduces the discomfort and risks associated with long-term wearing of orthokeratology lenses.

### BRIEF DESCRIPTION OF THE DRAWINGS

FIG. 1 is an exploded view of a photo-electric cooperative intervention orthokeratology lens of the disclosure;

FIG. 2 is a schematic structural diagram of the external transmitting terminal in the photo-electric cooperative intervention orthokeratology lens of the disclosure;

FIG. 3 is a schematic structural diagram of the light intervention unit in the photo-electric cooperative intervention orthokeratology lens of the disclosure;

FIG. 4 is a schematic structural diagram of the electric field intervention unit in the photo-electric cooperative intervention orthokeratology lens of the disclosure;

FIG. 5 is an equivalent circuit diagram of the photo-electric cooperative intervention orthokeratology lens of the disclosure;

FIG. 6 is a preparation flow chart of the internal receiving terminal in Embodiment 1;

FIG. 7 is a schematic diagram showing the external transmitting terminal arranged on an eyeglass frame in Embodiment 1;

FIG. 8 is a physical diagram of the internal receiving terminal in Embodiment 1; wherein, (a) is a side view; (b) is a front view in actual operation;

FIG. 9 is a schematic diagram of the animal model using the orthokeratology lens of Embodiment 1, as well as the light intervention and electric field intervention;

FIG. 10 is a therapeutic effect diagram of myopia treatment via light intervention and electric field intervention on a guinea pig myopia animal model using the devices provided in Embodiment 1; and

FIG. 11 is a time-dependent therapeutic effect diagram of myopia treatment via light intervention and electric field intervention on a guinea pig myopia animal model using the devices provided in Embodiment 1.

### DETAILED DESCRIPTION OF THE EMBODIMENTS

To clarify the objectives, technical solutions and advantages of the disclosure, the disclosure is described in further detail below with reference to the embodiments and the accompanying drawings.

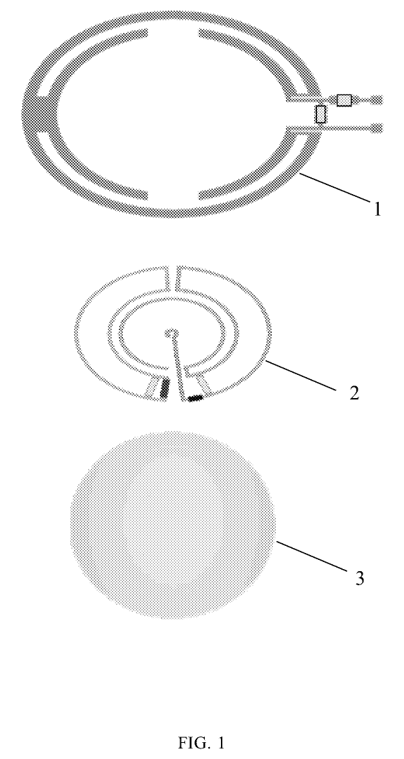

Referring to FIG. 1, a photo-electric cooperative intervention orthokeratology lens comprises an external power supply, an external transmitting terminal 1, an internal receiving terminal 2 and an orthokeratology lens body 3. The internal receiving terminal 2 is disposed on an outer surface of the orthokeratology lens body 3, and an inner surface of the orthokeratology lens body 3 is configured to contact an eyeball. The external transmitting terminal 1 is integrated on an eyeglass frame. The external power supply is configured to supply power to the external transmitting terminal 1 and is not shown in the figure.

Referring to FIG. 1, the internal receiving terminal 2 comprises a light intervention unit and an electric field intervention unit. The light intervention unit is configured to provide red light intervention, and the electric field intervention unit is configured to provide an electric field. The internal receiving terminal 2 is electromagnetically coupled with the external transmitting terminal 1 to provide wireless power supply to the light intervention unit and the electric field intervention unit.

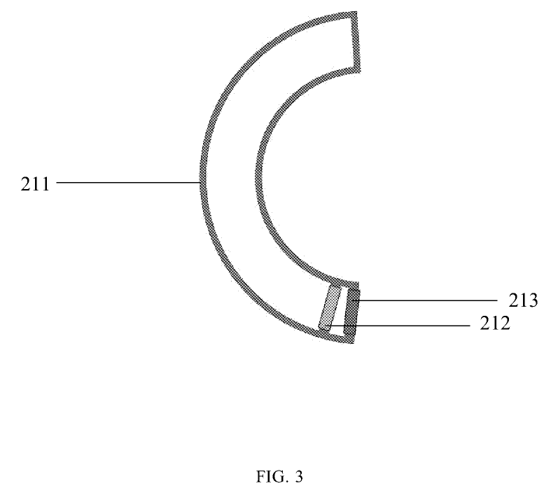

Referring to FIG. 3, the light intervention unit comprises a first metal coil 211, a first chip capacitor 212, and a red SMD LED 213. The first metal coil 211 comprises two concentric arcs and a first rectangular metal segment connecting the two concentric arcs. The first chip capacitor 212 is connected in parallel with the red SMD LED 213. The first chip capacitor 212 and the red SMD LED 213 are disposed between unconnected ends of the two concentric arcs. Two ends of the first chip capacitor 212 and the red SMD LED 213 are respectively connected to the two concentric arcs to form a series resonant circuit.

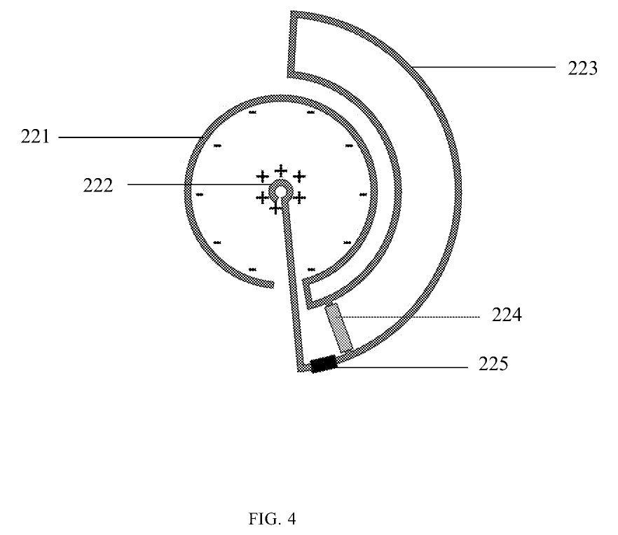

As shown in FIG. 4, the electric field intervention unit comprises a first electrode 221, a second electrode 222, a second metal coil 223, a second chip capacitor 224, and a rectifier 225.

The second metal coil 223 has the same structure as the first metal coil 211 and is symmetrically arranged with the first metal coil 211 to form a circular shape. The second chip capacitor 224 is connected in series at the unconnected ends of the two concentric arcs of the second metal coil 223.

The first electrode 221 comprises a first metal circle having a first opening, and a first end of the first opening is connected to an unconnected end of an inner arc of the second metal coil 223 through a second rectangular metal segment. A circle formed by the first metal coil 211 and the second metal coil 223 is concentric with the first electrode 221.

The second electrode 222 comprises a second metal circle having a second opening and an electrode wire connected to a first end of the second opening. A second end of the electrode wire is connected to a first end of the rectifier 225. A second end of the rectifier 225 is connected to an unconnected end of an outer arc of the second metal coil 223. As shown in FIG. 3, the electric field is positive inside and negative outside, and corneal epithelial cells move from the positive electrode to the negative electrode under electric field intervention.

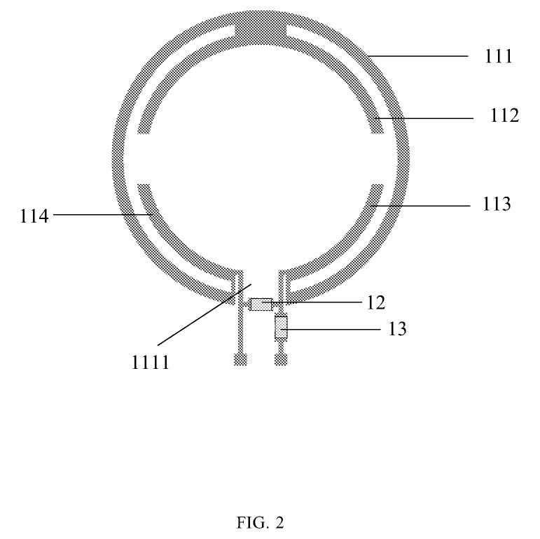

Referring to FIG. 2, the external transmitting terminal 1 comprises a first patterned metal layer, a third chip capacitor 12, and a fourth chip capacitor 13.

The first patterned metal layer comprises a third metal circle 111 having a third opening 1111, a semicircular metal strip 112, a first metal arc segment 113, and a second metal arc segment 114. The first metal arc segment 113 and the second metal arc segment 114 are of equal length. The first metal arc segment 113 and the second metal arc segment 114 are disposed adjacent to each other. The semicircular metal strip 112 is disposed on a first side of a diameter of the third metal circle 111, and the first metal arc segment 113 and the second metal arc segment 114 are disposed on a second side of the diameter. A center of the semicircular metal strip 112 is metallically connected to the third metal circle 111. A first end of the first metal arc segment 113 is connected to a first open end of the third metal circle 111 through a third rectangular metal segment, and a first end of the second metal arc segment 114 is connected to a second open end of the third metal circle 111 through a fourth rectangular metal segment. Two ends of the third chip capacitor are respectively connected to the first open end and second open end of the third metal circle 111. A first end of the fourth chip capacitor is connected to the third metal circle 111, and a second end of the fourth chip capacitor is connected to the external power supply.

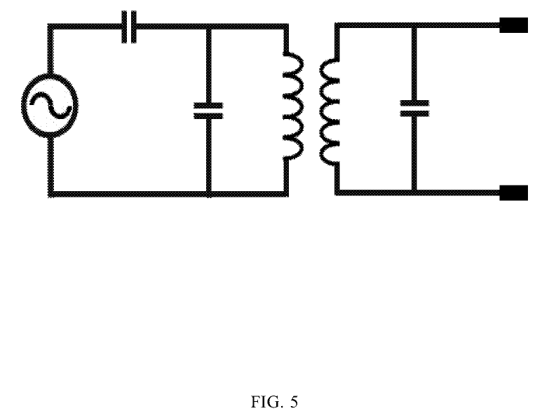

Referring to FIG. 5, the external transmitting terminal 1 on the left side comprises a transmitting resonant circuit. The transmitting resonant circuit comprises an AC power supply, a transmitting capacitor, and a transmitting coil. The AC power supply drives the transmitting coil to generate an alternating magnetic field. The transmitting coil and a receiving coil of the internal receiving terminal 2 on the right side achieve electromagnetic coupling through mutual inductance, thereby wirelessly transmitting energy in the form of a magnetic field. The receiving coil and a receiving capacitor at the internal receiving terminal 2 form a receiving resonant circuit. Power from the receiving resonant circuit is supplied to the electric field intervention unit and the light intervention unit through an output terminal.

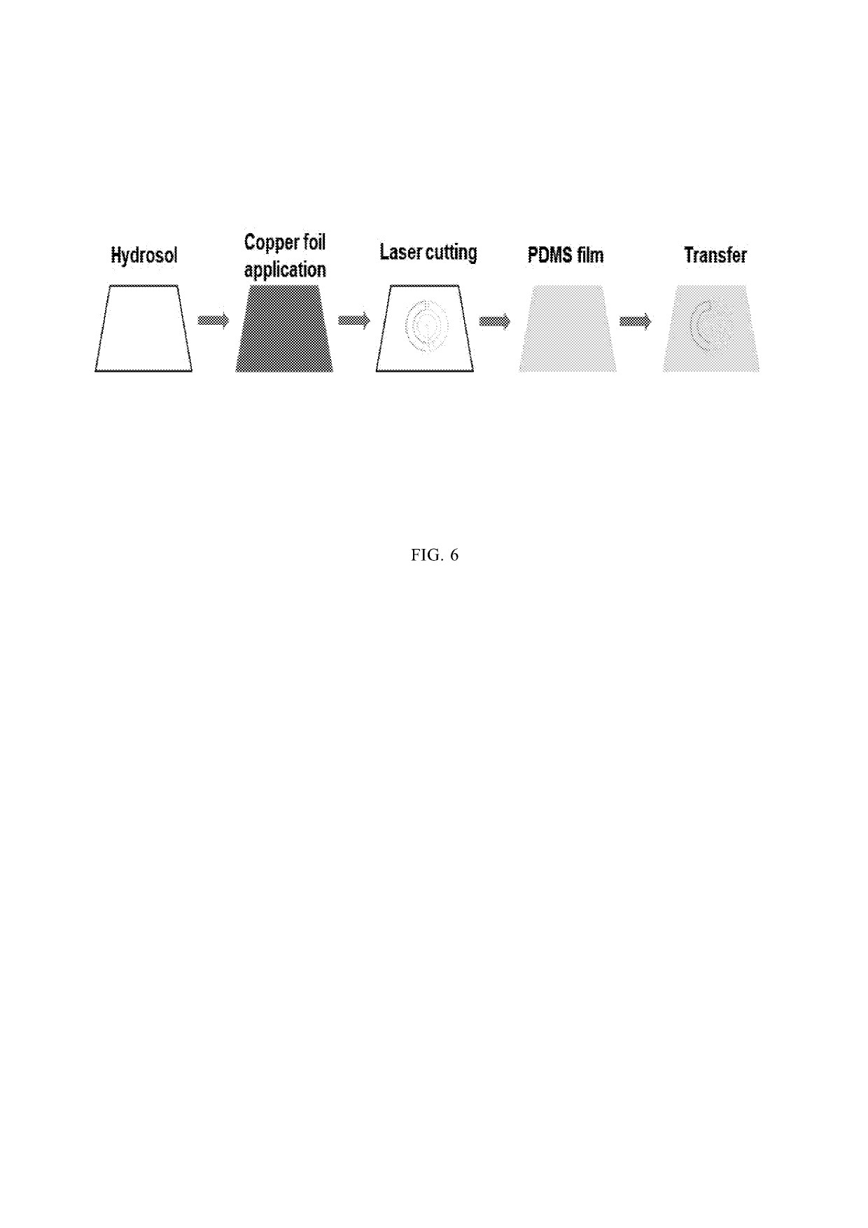

FIG. 6 is a preparation flow chart of the internal receiving terminal 2 in Embodiment 1. Firstly, a copper film is attached to a water-soluble adhesive tape; the copper film is cut into a coil structure of a specific shape by femtosecond laser cutting technology; subsequently, PDMS liquid A and liquid B are mixed at a ratio of 1:10 and stirred evenly to form a homogeneous mixture; 1.5 mL of the mixture is dropped onto a glass slide and spin-coated at 1000 rpm for 20 seconds; after heating at 60°C for 20 minutes, the upper layer of the metal coil is transferred to the PDMS material, and then transferred to the surface of the orthokeratology lens through PDMS transfer printing.

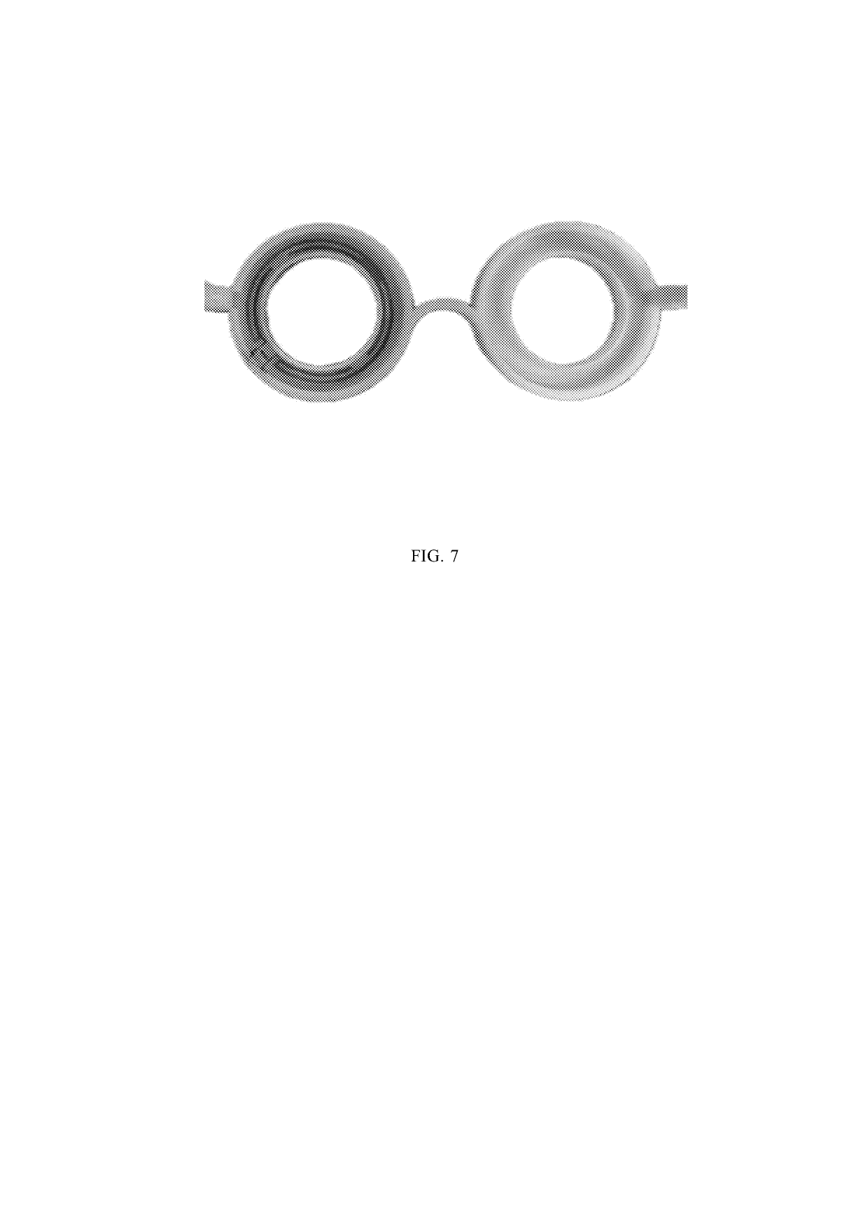

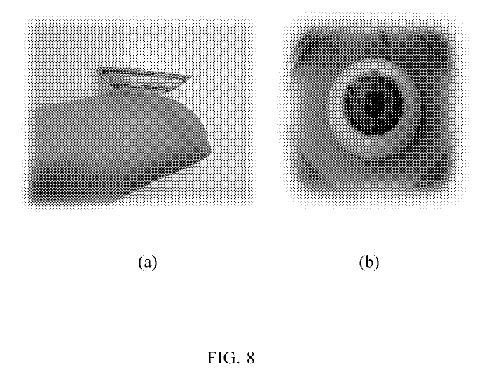

The external transmitting terminal 1 is disposed on an eyeglass frame, as shown in FIG. 7. The external transmitting terminal 1 and the internal receiving terminal 2 constitute an electromagnetic coupling energy transmission system. The transmitting coil generates the magnetic field, and the receiving coil induces an alternating current after sensing the magnetic field, supplying power to the electric field intervention unit and the light intervention unit to realize electric field stimulation and LED lighting. In operation, the transmitting capacitor and the receiving capacitor are configured to match the resonant frequencies of the transmitting resonant circuit and the receiving resonant circuit within a range of 40-50 MHz. This frequency matching maximizes electromagnetic coupling efficiency between the transmitting coil and the receiving coil. The wirelessly transmitted energy powers the light intervention unit and supplies electric field intervention unit to generate the electric field for corneal epithelial cell migration. A physical diagram of the internal receiving terminal 2 is shown in FIG. 8.

### Embodiment 1

A photo-electric cooperative intervention orthokeratology lens for myopia improvement, the preparation parameters of each component are as follows.

The external transmitting terminal comprises a transmitting resonant circuit. The transmitting resonant circuit comprise a transmitting coil, a third chip capacitor, and a fourth chip capacitor. The third chip capacitor is connected in parallel with the transmitting coil. The fourth chip capacitor is connected in series with the transmitting coil. The transmitting coil is a multi-turn copper wire coil having a wire diameter of 0.25 mm, an outer diameter of the coil of 45 mm, and an inner diameter of 40 mm. The fourth chip capacitor has a capacitance value of 10 pF, and the third chip capacitor has a capacitor of 1 nF. The third chip capacitor and the fourth chip capacitor are configured to adjust the resonant frequency of the transmitting resonant circuit to a range of 40-50 MHz. In the LC circuit formed by the transmitting coil, the third chip capacitor, and the fourth chip capacitor, the connection mode of the third chip capacitor and the fourth chip capacitor affect the overall resonant frequency: when a capacitor is connected in parallel, the total capacitance increases, energy exchange slows down, and the resonant frequency decreases; when a capacitor is connected in series, the total capacitance decreases, energy exchange speeds up, and the resonant frequency increases.

The internal receiving terminal 2 comprises a receiving resonant circuit, comprising a light intervention unit and an electric field intervention unit. The light intervention unit comprises a first metal coil, a first chip capacitor, and a red SMD LED. The first chip capacitor 212 is connected in parallel with the first metal coil 211. The first metal coil is a single-turn copper wire coil having a wire diameter of 0.2 mm, an outer arc diameter of 12 mm, and an inner arc diameter of 8 mm. The first chip capacitor has a capacitance of a 560 pF. The red SMD LED has a wavelength range of 650 nm–680 nm and an irradiance of 0.1–2.0 mW/cm².

The electric field intervention unit comprises a second metal coil 223, a second chip capacitor 224, a first electrode 221, and a second electrode 222. The second chip capacitor 224 is connected in series with the second metal coil 223. The second metal coil is a single-turn copper wire coil having a wire dimeter of 0.2 mm, a loop diameter of 12 mm, an outer arc diameter of 12 mm, and an inner arc diameter of 8 mm. The first electrode 221 comprise a first metal circle having a first opening, and the first metal circle has a diameter of 7 mm. The second electrode 222 comprises a second metal circle having a second opening, and the second metal circle has a diameter of 1 mm. The second chip capacitor 224 has a capacitance of 560 pF.

The photo-electric cooperative intervention orthokeratology lens of Embodiment 1 was used to conduct photo-electric intervention treatment on a guinea pig myopia model as follows.

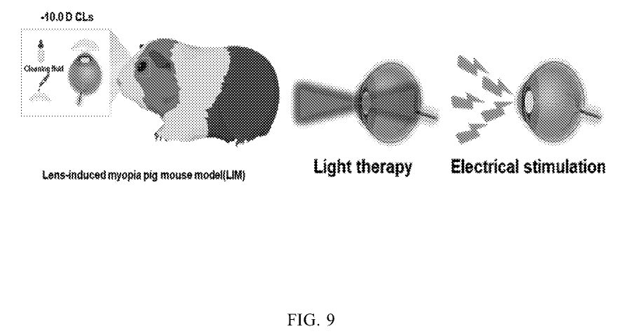

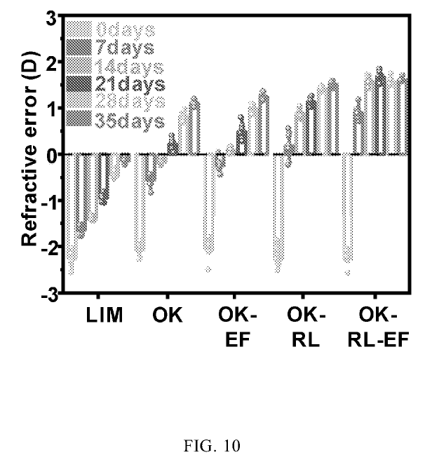

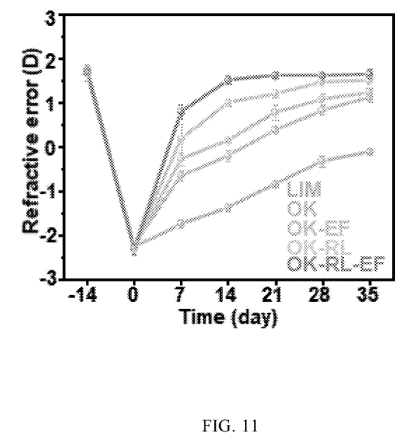

As shown in FIG. 9, to induce a lens-induced myopia (LIM) model, guinea pigs were fitted with custom-made monocular rigid gas-permeable contact lenses with a diopter of -10 for a 2-week modeling period. As shown in FIG. 10 and FIG. 11, the diopter of both left and right eyes of the LIM guinea pig model reached -2 D. At day-14 (before modeling), the eyes were normal with an average diopter of about +1.7 D; after 2 weeks of modeling, the average diopter reached about -2.5 D, indicating that the guinea pigs developed moderate myopia, meeting the requirements of the myopia model. After modeling, the LIM guinea pig models were divided into 5 groups with 4 guinea pigs in each group (n=4): a control group (LIM), an orthokeratology group (hereinafter referred to as “OK group”), an orthokeratology with electric field intervention group (hereinafter referred to as “OK-EF group”), an orthokeratology with red light intervention group (hereinafter referred to as “OK-RL group”), and an orthokeratology with combined red light and electric field intervention group of the present disclosure (hereinafter referred to as “OK-RL-EF group”). Each intervention lens was worn for one hour daily for 35 days. For the control group (LIM) , no treatment was applied. After 35 days, the myopia of the guinea pigs was slightly alleviated by about -0.8 D, failing to return to the normal level of about +1.7 D. For the OK group, the diopter improved to about 0.9 D after 35 days, not reaching the normal level. For the OK-EF group, the diopter recovered to about +1 D after 35 days, which was still below the normal level. For the OK-RL-EF group, the diopter restored to about +1.2 D after 35 days, not returning to normal. For the OK-RL-EF group, the diopter recovered to about +1.5 D after 14 days, approaching the normal level of about +1.7 D.

The above descriptions are merely specific implementations of the disclosure. Any feature disclosed in this specification can be replaced by other equivalent features or features with similar purposes unless specifically stated otherwise; all disclosed features, or steps in all methods or processes, can be combined in any way except for mutually exclusive features and/or steps.

What is claimed is:

1. A photo-electric cooperative intervention orthokeratology lens for myopia improvement, comprising:
an external power supply;
an external transmitting terminal;
an internal receiving terminal; and
an orthokeratology lens body;
wherein:
the internal receiving terminal is disposed on an outer surface of the orthokeratology lens body, and an inner surface of the orthokeratology lens body is configured to contact an eyeball;
the external transmitting terminal is integrated on an eyeglass frame;
the external power supply is connected to the external transmitting terminal;
the internal receiving terminal comprises a light intervention unit and an electric field intervention unit;
the light intervention unit is configured to provide red light intervention, and the electric field intervention unit is configured to provide an electric field; and
the internal receiving terminal is electromagnetically coupled with the external transmitting terminal to provide wireless power supply to the light intervention unit and the electric field intervention unit.

2. The photo-electric cooperative intervention orthokeratology lens of claim 1, wherein the electromagnetic coupling operates at a resonant frequency band of 40–50 MHz.

3. The photo-electric cooperative intervention orthokeratology lens of claim 1, wherein the light intervention unit comprises:
a first metal coil comprising two concentric arcs and a first rectangular metal segment connecting the two concentric arcs;
a first chip capacitor, and
a red SMD LED;
wherein:
the first chip capacitor is connected in parallel with the red SMD LED, the first chip capacitor and the red SMD LED are disposed between unconnected ends of the two concentric arcs, and two ends of the first chip capacitor and the red SMD LED are respectively connected to the two concentric arcs to form a series resonant circuit.

4. The photo-electric cooperative intervention orthokeratology lens of claim 3, wherein the red light emitted by the red SMD LED has a wavelength range of 650 nm–680 nm.

5. The photo-electric cooperative intervention orthokeratology lens of claim 3, wherein a power density of the red SMD LED is 0.1–2.0 mW/cm².

6. The photo-electric cooperative intervention orthokeratology lens of claim 3, wherein the electric field intervention unit comprises:
a first electrode;
a second electrode;
a second metal coil;
a second chip capacitor; and
a rectifier;
wherein:
the second metal coil has the same structure as the first metal coil and is symmetrically arranged with the first metal coil to form a circular shape; the second chip capacitor is connected in series between the unconnected ends of the two concentric arcs of the second metal coil;
the first electrode comprises a first metal circle having a first opening, and a first end of the first opening is connected to an unconnected end of an inner arc of the second metal coil through a second rectangular metal segment; a circle formed by the first metal coil and the second metal coil is concentric with the first electrode; and
the second electrode comprises a second metal circle having a second opening and an electrode wire connected to a first end of the second opening; a second end of the electrode wire is connected to a first end of the rectifier; and a second end of the rectifier is connected to an unconnected end of an outer arc of the second metal coil.

7. The photo-electric cooperative intervention orthokeratology lens of claim 6, wherein the second electrode comprises an ITO transparent electrode material.

8. The photo-electric cooperative intervention orthokeratology lens of claim 1, wherein the external transmitting terminal comprises:
a first patterned metal layer;
a third chip capacitor; and
a fourth chip capacitor;
wherein:
the first patterned metal layer comprises a third metal circle having a third opening, a semicircular metal strip, a first metal arc segment, and a second metal arc segment;
the first metal arc segment and the second metal arc segment are of equal length;
the first metal arc segment and the second metal arc segment are disposed adjacent to each other;
the semicircular metal strip is disposed on a first side of a diameter of the third metal circle, and the first metal arc segment and the second metal arc segment are disposed on a second side of the diameter;
a center of the semicircular metal strip is metallically connected to the third metal circle;
a first end of the first metal arc segment is connected to a first open end of the third metal circle through a third rectangular metal segment; and a first end of the second metal arc segment is connected to a second open end of the third metal circle through a fourth rectangular metal segment; two ends of the third chip capacitor are respectively connected to the first open end and second open end of the third metal circle; and
a first end of the fourth chip capacitor is connected to the third metal circle and a second end of the fourth chip capacitor is connected to the external power supply.

9. The photo-electric cooperative intervention orthokeratology lens of claim 8, wherein the external power supply comprises a function signal generator.

10. The photo-electric cooperative intervention orthokeratology lens of claim 8, wherein the first chip capacitor and the second chip capacitor have a same capacitance value, and the capacitance values of the first chip capacitor, the third chip capacitor and the fourth chip capacitor are different from each other to enable electromagnetic coupling between the external transmitting terminal and the internal receiving terminal.

11. The photo-electric cooperative intervention orthokeratology lens of claim 10, wherein:
the capacitance value of the first chip capacitor is 120–680 pF;
the capacitance value of the second chip capacitor is 120–680 pF;
the capacitance value of the third chip capacitor is 1–10 nF; and
the capacitance value of the fourth chip capacitor is 10–100 pF.

12. The photo-electric cooperative intervention orthokeratology lens of claim 8, wherein the first metal coil and the second metal coil are formed by laser cutting copper foil.

13. The photo-electric cooperative intervention orthokeratology lens of claim 12, wherein a thickness of the copper foil is 10–30 μm.
